(19) United States
(12) Patent Application Publication
(10) Pub. No.: US 2020/0405770 A1
(43) Pub. Date: Dec. 31, 2020

(54) ADMINISTRATION OF FIBROBLASTS AND DERIVATIVES THEREOF FOR TREATMENT OF TYPE 2 DIABETES

(71) Applicant: Figene, LLC, Houston, TX (US)

(72) Inventors: Pete O'Heeron, Houston, TX (US); Thomas Ichim, San Diego, CA (US)

(73) Assignee: Figene, LLC, Houston, TX (US)

(21) Appl. No.: 16/913,860

(22) Filed: Jun. 26, 2020

Related U.S. Application Data
(60) Provisional application No. 62/867,976, filed on Jun. 28, 2019.

(51) Int. Cl. A61K 35/33; A61K 9/00; A61P 3/10

(52) U.S. Cl. CPC A61K 35/33; A61P 3/10; A61K 9/0019

(57) ABSTRACT

Embodiments of the disclosure encompass methods of increasing insulin sensitivity in an individual in need thereof. The increase in insulin sensitivity may derive from individuals that have diabetes, aging, low grade inflammation, obesity, pregnancy, metabolic syndrome X, congenital abnormality or a combination thereof. In specific embodiments, the methods encompass providing to an individual an effective amount of fibroblast cells of certain kinds.

### CROSS-REFERENCE TO RELATED APPLICATIONS

This application claims priority to U.S. Provisional Patent Application 62/867,976, filed Jun. 28, 2019, which is incorporated by reference herein in its entirety.

### TECHNICAL FIELD

Embodiments of the disclosure include at least the fields of cell biology, molecular biology, cell therapy, and medicine.

### BACKGROUND

Diabetes is a disease of hyperglycemia. There are two main forms of diabetes: Type 1 diabetes and Type 2. In Type 1 diabetes, also known as insulin-dependent diabetes mellitus (IDDM), or juvenile diabetes, the patient's pancreas produces little or no insulin, believed to be in part the result of autoimmune attached on the insulin producing beta-cells in the pancreas. It's one of the most costly, chronic diseases of childhood and one you never outgrow. It is believed that more than one million Americans have IDDM. Patients with full-blown IDDM must take multiple insulin injections daily or continually infuse insulin through a pump, and test their blood sugar by pricking their fingers for blood six or more times per day. Neither dietary therapy nor treatment with an oral hypoglycemic agent is effective, and only treatment with insulin is effective. Ketonemia and acidosis due to the loss of insulin secreting capacity, and if untreated, may result in diabetic coma. Because numerous factors such as stress, hormones, growth, physical activity, medications, illness/infection, and fatigue effect insulin utilization, even a strictly monitored program of insulin administration does not mimic the endogenous functions of the pancreas, and as a result numerous complications develop.

Type 2 diabetes, also known as Non-Insulin Dependent Diabetes Mellitus (NIDDM), or adult-onset diabetes, is associated with impairment of peripheral tissue response to insulin. NIDDM is believed to afflict approximately 18.2 million people in the US and as a result of the obesity epidemic, substantially younger patients are beginning to be diagnosed with this condition. The economic burden of NIDDM is witnessed in statistics demonstrating that on average, the health care costs for NIDDM patients are expensive.

Insulin resistance is present in almost all obese individuals W. However, compensatory insulin production by beta-cells usually occurs, thus preventing hyperglycemia. In response to prolonged insulin resistance, as well as other factors, beta cell insulin production eventually lose ability to cope with the increasing insulin demands and postprandial hyperglycemia occurs, characterizing the transition between normal glucose tolerance and abnormal glucose tolerance. Subsequently, the liver starts secreting glucose through hepatic gluconeogenesis (generation of glucose from substrates that are not sugars, not from glycogen) and hyperglycemia is observed even in the fasting state. In contrast to IDDM, NIDDM presents only a small degree of ketonemia and acidosis although the insulin action is reduced from normal, and treatment with insulin is not always required.

The greatest clinical challenge in this disease is the prevention of the long-term complications, many of which involve vascular, ocular and renal systems. Although various agents are utilized to increase glucose sensitivity, or to stimulate insulin secretion, these approaches are not optimal because they do not exactly mimic the physiological control of post-prandial insulin secretion. Accordingly, the fluctuations of glucose, as well as downstream metabolic consequences end up causing macrovascular pathology such as coronary atherosclerosis, and increased risk of stroke, as well as microvascular pathology such as macular degeneration and renal failure. Additionally, neuropathies are often present associated with hyperglycemia.

There are numerous treatments available for NIDDM; these depend on patient-specific characteristics, as well as severity of disease. The treatment goal in diabetes treatment is to bring plasma glucose levels down to as near normal levels, for example 80-120 milligrams per deciliter (mg/dl) before meals and 100-140 mg/dl at night. Numerous medical tests are known in the art for monitoring glucose, as well as cholesterol and lipid levels. The goal of maintaining normal glucose levels is judged in some ways by the ability to prevent secondary complications, such as retinopathy, neuropathy, vascular disease, and strokes.

In beginning phases of NIDDM, patients may be treated with various oral drugs, and as diabetes progresses, various forms of insulin may be administered. Although tight glucose control is known to decrease the rate of diabetic complications, such control is very difficult to achieve, and when it is achieved significant morbidity and mortality still occurs. Below are listed some of the non-insulin treatments for NIDDM.

Mainstream oral treatments for diabetes can be separated by mechanism of action into two groups: hypoglycemics, such as sulfonylureas and meglitinides which induce beta cell insulin secretion; and antihyperglycemics such as biguanides and alpha-glucosidase inhibitors that cause uptake of glucose.

Sulfonylureas are a type of drug that stimulate insulin release from beta cells. Essentially, these agents work by blocking ATP-sensitive potassium channels in the pancreatic beta-cell membrane. This effect is mediated by the binding of the drug to the sulfonylurea receptor (SUR) subunit of the channel Inhibition of the potassium channel leads to depolarization of the cell membrane and insulin secretion, in a similar way as if glucose was added to the cell. Glyburide is a second generation sulfonylurea compound that is sold under the names Micronase, DiaBeta, or Glynase. Glipizide, sold under the names Glucotrol and Glucotrol XL, is also a second generation sulfonylurea drug. Third-generation sulfonylurea drugs include Glimepiride (Amaryl). This agent is believed to have greater safety in patients with ischemic heart disease as compared to other sulfonylurea drugs. Glimepiride is the only sulfonylurea based drug that is approved for use together with insulin or metformin. In general, sulfonylurea drugs suffer from the disadvantage that the amount of insulin secretion induced depends on the timing and dose of drug administration and not by the blood glucose levels. This causes not only various fluctuations in glucose level but also digestive symptoms such as anorexia in some patients.

Meglitinides (commonly called glinides) are a class of insulin secretagogues that have short-acting activity, given after meals. Similar to sulfonylurea drugs in that mechanistically they induce insulin secretion by closure of the ATP-dependent potassium channel, glinides appear to be more short-term in activity. Theoretically these drugs have less risk of inducing hypoclycemia and cause a physiological-like insulin release pattern. Repaglinide, sold under the name Prandin, and Nateglinide, sold under the name Starlix, are examples of two glinides. When compared with sulfonylurea drugs, glinides have been shown to provide a better control of postprandial hyperglycaemia, not to induce hypoglycaemia, and to generally have better safety profile, especially in patients with renal failure [2].

Biguanides are a class of drugs that decrease hepatic glucose production and increase insulin sensitivity. Metformin, sold under the names Glucophage, Glucophage XR, and Metformin XR is an example of a biguanide. It is also the most widely prescribed oral antidiabetic in the world and is in most circumstances the agent of choice for first line initial therapy of the typical obese patient with type 2 DM and mild to moderate hyperglycaemia [3]. Metformin administration is associated with weight loss and improvement in lipid profile. Metformin is effective as monotherapy and, in combination with both insulin secretagogues and thiazolidinediones (TZDs), may alleviate the need for insulin treatment [4]. It is known that metformin induces increased glucose utilization and reduction in leptin concentrations [5]. Additionally, metformin induces inhibition of dipeptidyl peptidase-IV activity, which allows for extended half-life of GLP-1 [6]. Classical mechanisms of action include increased glucose use by anaerobic glycolysis, inhibition of hepatic gluconeogenesis, and suppression of intestinal absorption of glucose. One adverse effect associated with various biguanides is lactic acidosis.

Thiazolidinediones (glitazones) are a family of drugs that decrease insulin resistance in both muscle and adipose tissue. They do not induce insulin secretion. Rosiglitazone, sold under the name Avandia, and Pioglitazone, sold under the name Actos are two thiazolidinediones. These agents induce insulin sensitivity through the activation of insulin receptor kinase, thereby promoting glucose uptake by peripheral tissues, and ameliorating increased liver glucose production. Known side effects include digestive symptoms and edema, and hematological alterations, and upregulation in plasma LDH. Glitazones are interesting not only from their ability to increase insulin signal transduction, but also due to anti-inflammatory effects. It is known, for example, that rosiglitazone inhibits ability of dendritic cells to secrete interleukin-12 after stimulation via CD40 [7]. This is believed to occur via activation of PPAR-gamma pathways. Additionally, treatment with rosiglitazone is able to inhibit onset of colitis in animal models through preferential induction of Th2 cytokine production [8].

Alpha-glucosidase inhibitors are used to delay rate of sugar absorption. Acarbose, sold under the name Precose, and Miglitol sold under the name Glyset are two examples of drugs in this family.

Incretin mimetics mirror glucose-dependent insulin secretion, cause inhibition of glucagon secretion, and delay gastric emptying. Exenatide, sold under the name Byetta, is a glucagon-like-peptide-1 (GLP-1) receptor agonist and stimulates insulin secretion from the beta cell. Controlled clinical trials provided evidence that glycaemic control under exenatide administered twice daily in a dose of 5-10 micrograms was not inferior to conventional insulin therapy.

Currently available treatments for NIDDM lack the capability of mimicking an endogenous insulin secretion and insulin utilization response. Accordingly various approaches have been pursued aimed at utilization of cell therapy for generating synthetic islets. These approaches have included U.S. Pat. No. 7,056,734 that discloses the ability of GLP and Exendin-4 to induce differentiation of cells into insulin produce or amylase producing cells. The patent covers use of GLP-1 or related molecules to make either non-insulin producing cells, or amylase producing cells, into insulin producing cells, as well as using Exendin-4 for making either non-insulin producing cells, or amylase producing cells, into insulin producing cells.

U.S. Pat. No. 6,903,073 addresses the stimulation of hedgehog expression to increase insulin production. This is based on findings that inhibiting hedgehog signaling reduces insulin production, and transfection with hedgehog increases insulin production [9].

U.S. Pat. No. 6,967,019 discloses ways of making gastrointestinal organ cells and pancreatic cells express insulin in vitro, conceptually for introduction in vivo. The patent essentially teaches that introduction of a neuroendocrine class B basic helix-loop-helix (bHLH) transcription factor gene or the neurogenin3 (Ngn3) gene into gastrointestinal organ cells or pancreatic cells, respectively, endows ability to produce insulin. Unfortunately, no evidence of glucose regulation was provided.

U.S. Pat. No. 7,033,831 shows a method of generating insulin producing cells from human embryonic stem cells through the process of first incubating the human embryonic stem cells with Activin A, and then subsequently incubating the cells with nicotinamide. Activin is a peptide involved in wound healing and morphogenesis, whereas nicotinamide is a type of vitamin B3 and improves beta cell functions. The patent covers the culturing of ES cells first in Activin A, then nicotinamide as a method of generating insulin producing cells. Also covered are methods of producing insulin secreting cells, through first growing embryoid bodies, then treating the embryoid bodies with a TGF-b antagonist together with one or more mitogens (to stimulate proliferation), and subsequently culturing the cells in nicotinamide. Additionally covered is the use of embryonic stem cells and not embryoid bodies as starting tissue for generation of insulin producing cells.

U.S. Pat. No. 7,169,608 describes a simple method of inducing differentiation of bone marrow into islets by a simple two step culture approach involving an initial culture in low concentration of glucose (at least 3 days) followed by a subsequent culture in high concentration of glucose (at least 7 days). According to the patent, the resulting cells generate insulin in response to sugar, and are capable of preventing diabetes when administered in vivo into animals. The patent is interesting because authors have actually published some of the data from the patent [10]. Noteworthy points about the published data is that the bone marrow derived cells appear to take an architecture similar to that found in the normal islets when administered in vivo. The transplanted cells produce insulin (I and II), glucagon, somatostatin and pancreatic polypeptide, and C-peptide. In addition, various animal models of diabetes were cured by administration of bone marrow cells that were manipulated according to the invention.

U.S. Pat. No. 7,138,275 provides that culturing of peripheral blood monocytes in the presence of IL-3 and M-CSF for approximately 6 days, induces a program of de-differentiation in the monocytes to endow them with stem cell like potential. The patent goes on to demonstrate that these monocytes can be converted into islets, and shows efficacy in a streptozocin-treated diabetic mouse model of diabetes.

For the above patents it is obvious that although some generation of insulin producing cells was reported in vitro, and in some cases, in vivo, therapeutic applications of this is limited. In NIDDM, the high insulin demands needed to overcome insulin resistance place significant stress on the beta cell. This “need” for hyperinsulin production, as well as other factors associated with hyperglycemia, often lead to accelerated beta cell apoptosis through mechanisms such as Fas, the ATP-sensitive K+ channel, insulin receptor substrate 2, oxidative stress, nuclear factor-kappaB, endoplasmic reticulum stress, and mitochondrial dysfunction [11]. Thus, even if an appropriate beta cell source could be generated as described in the above patents, it is unlikely to yield long-term beneficial clinical results because of the underlying causative elements that initiated diabetes onset originally.

The present disclosure provides solutions to a long-felt need in the art for Type 2 diabetes treatment and prevention.

### BRIEF SUMMARY

Embodiments of the disclosure relate to the field of metabolic diseases and treatment or prevention thereof. In particular embodiments, the disclosure provides methods of treating insulin resistance and providing an environment suitable for restoration of insulin-producing cell function. In certain embodiments, the disclosure encompasses methods of treating and/or preventing insulin resistance using cell therapy and in at least certain cases includes fibroblast cell therapy and, in some embodiments, combinations of fibroblasts with one or more various pharmacological and medical interventions. Methods of treating Type 2 diabetes are included herein, including reducing the severity and/or delaying the onset of it. Methods also included concern restoring insulin producing cell function. Embodiments of the disclosure concern methods of preventing, delaying, or reducing the severity of one or more complications from Type 2 diabetes. Methods encompassed herein also include methods of increasing insulin sensitivity, methods of keeping blood glucose at normal levels (50-110 mg/dL), methods of increasing skeletal muscle perfusion, methods of endowing insulin responsiveness, methods of reducing inflammatory mediators, and so forth. In at least some embodiments, the methods and compositions utilized herein are not for Type I diabetes.

Embodiments of the disclosure encompass methods of treating or preventing insulin resistance in an individual, comprising the step of delivering to the individual a therapeutically effective amount of fibroblast cells. The fibroblasts may be CD105+, CD34+, CD133+, or a mixture thereof. The fibroblasts may additionally or alternatively be CD90+, CD45− and/or CD14−. In particular embodiments, the fibroblasts have regenerative activity. In certain cases, the fibroblasts have been exposed to erythropoietin, prolactin, human chorionic gonadotropin, gastrin, EGF, FGF, and/or VEGF, and in some cases this results in the fibroblasts having regenerative activity. In particular cases, the insulin resistance is the result of diabetes, aging, low grade inflammation, obesity, pregnancy, metabolic syndrome X, congenital abnormality, or a combination thereof. Fibroblasts utilized in any methods herein may be derived from cord blood, peripheral blood, menstrual blood, placental matrix, endometrium, umbilical cord blood, deciduous teeth, muscle tissue, placenta, skin, bone marrow, amniotic fluid, adipose, umbilical cord matrix, omentum, subintestinal mucosa, or a mixture thereof. The fibroblast cells may possess the ability to proliferate at a rate of more than one double per 24 hours when cultured at a concentration of 20,000 cells per well in a 96 well plate in 10% fetal calf serum in DMEM media. The fibroblast cells may be delivered to the individual systemically or locally. The fibroblast cells may be delivered to the individual intramuscularly, and/or the fibroblast cells may be delivered to the individual into or near the pancreas. In some cases, the methods further comprise the step of providing to the individual a therapeutically effective amount of one or more anti-inflammatory agents and/or the method may further comprise the step of providing to the individual a therapeutically effective amount of one or more diabetes therapies of any kind.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE FIGURES

FIG. 1 shows blood glucose levels in mice models of diabetes that were given control (saline), bone marrow MSCs, adipose MSC, or fibroblasts (from left to right in the bar groupings).

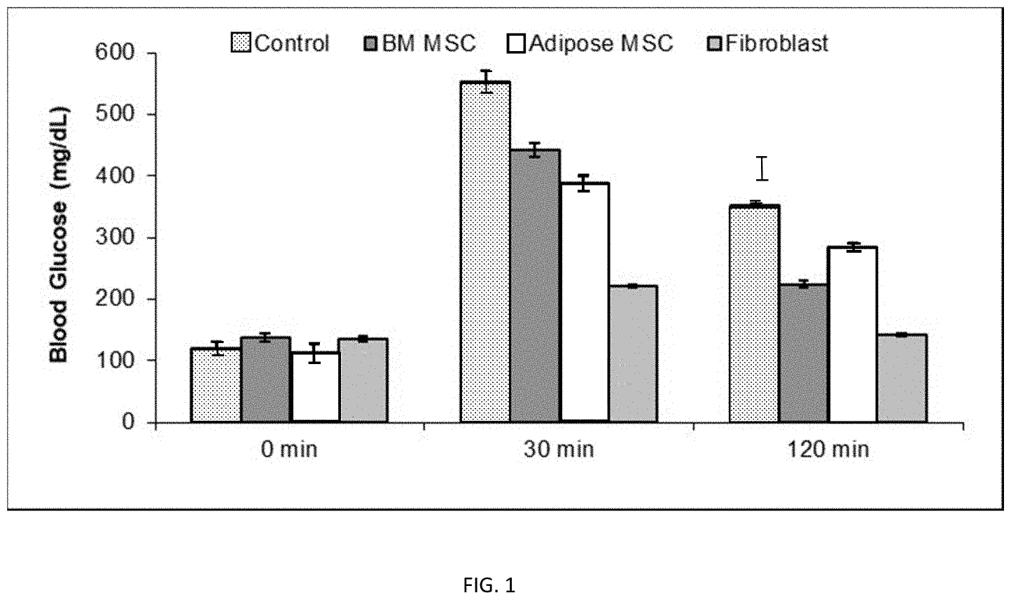

### DETAILED DESCRIPTION

### I. Examples of Definitions

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the invention, for example. Some embodiments may consist of or consist essentially of one or more elements, method steps, and/or methods of the invention. It is contemplated that any method or composition described herein can be implemented with respect to any other method or composition described herein. The scope of the present application is not intended to be limited to the particular embodiments of the process, machine, manufacture, composition of matter, means, methods and steps described in the specification.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe, etc. A second exemplary method of administering is by a direct mechanism such as, local tissue administration, oral ingestion, transdermal patch, topical, inhalation, suppository, etc.

The term “allogeneic,” as used herein, refers to cells of the same species that differ genetically from cells of a host.

The term “autologous,” as used herein, refers to cells derived from the same subject. The term “engraft” as used herein refers to the process of stem cell incorporation into a tissue of interest in vivo through contact with existing cells of the tissue.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “activated fibroblasts” refers to fibroblasts treated with one or more agents and/or stimuli capable of inducing one or more alterations in the cell: metabolic, immunological, growth factor-secreting, surface marker expression, and/or production of microvesicles. Examples of agents include epidermal growth factor (EGF; (Peprotech), Transforming Growth Factor-alpha (TGF-alpha; Peprotech), basic Fibroblast Growth Factor (bFGF; Peprotech), brain-derived neurotrophic factor (BDNF; R&D Systems), and Keratinocyte Growth Factor (KGF; Peprotech). EGF is a potent mitogenic factor for a variety of cultured ectodermal and mesodermal cells and has a profound effect on the differentiation of specific cells in vivo and in vitro and of some fibroblasts in cell culture. The EGF precursor exists as a membrane-bound molecule which is proteolytically cleaved to generate the 53-amino acid peptide hormone that stimulates cells. A preferred mitogenic growth factor is EGF. EGF is preferably added to the basal culture medium at a concentration of between 5 and 500 ng/ml or of at least 5 and not higher than 500 ng/ml. A preferred concentration is at least 10, 20, 25, 30, 40, 45, or 50 ng/ml and not higher than 500, 450, 400, 350, 300, 250, 200, 150, or 100 ng/ml. A more preferred concentration is at least 50 and not higher than 100 ng/ml. An even more preferred concentration is about 50 ng/ml or 50 ng/ml. The same concentrations could be used for a FGF, preferably for FGF10 or FGF7. If more than one FGF is used, for example, FGF7 and FGF10, the concentration of a FGF is as defined above and refers to the total concentration of FGF used. During culturing of stem cells, the mitogenic growth factor is preferably added to the culture medium every second day, while the culture medium is refreshed preferably every fourth day. Any member of the bFGF family may be used. In some cases, FGF7 and/or FGF10 is used. FGF7 is also known as KGF (Keratinocyte Growth Factor).

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of one or more of nutrients, growth factors, and the presence of other components. The growth factors used to supplement media are often derived from animal blood, such as calf serum.

As used herein, the term “conditioned medium of fibroblast regenerative cells” refers to a liquid media that has been in contact with cells, wherein the cells produce one or more factors that enter the media, thus bestowing upon the media at least one therapeutic activity.

The term “fibroblast derivative” as used herein refers to a dedifferentiated fibroblast or an apoptotic body or an exosome derived from a fibroblast.

The term “individual”, as used herein, refers to a human or animal that may or may not be housed in a medical facility and may be treated as an outpatient of a medical facility. The individual may or may not be receiving one or more medical compositions from a medical practitioner and/or via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method and/or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “prevent” or “preventing” refers to a method wherein a medical condition or onset of at least one symptom thereof is kept from occurring.

The term “subject” or “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to promote formation of new blood vessels and associated vasculature (angiogenesis) and/or an amount sufficient to promote repair or remodeling of existing blood vessels and associated vasculature. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

### II. Methods of Manufacture and Use

Disclosed are methods, compositions, and cells useful for increasing insulin sensitivity and/or improving lack of insulin production in a host in need thereof. One aspect of the disclosure encompasses methods of increasing skeletal muscle perfusion through administration of cells capable of directly and/or indirectly stimulating angiogenesis and/or vascular responsiveness by administration of fibroblasts and/or fibroblast-like cells and/or microvesicles thereof. Another aspect provides means of increasing sensitivity to insulin through administration of a cell composition capable of integrating into host insulin-responsive tissue and upregulating responsiveness either through mobilization of host cells capable of responding to insulin, mobilization of host cells capable of endowing insulin responsiveness on other host cells, exogenously administered cells taking the role of insulin responsiveness, and/or exogenously administered cells endowing insulin responsiveness on other host cells. Another aspect comprises modifying the host to allow for concurrent insulin sensitization and upregulated production of insulin. Encompassed herein are methods of treating Type 2 diabetes, including reducing the severity and/or delaying the onset of it. Methods also included concern restoring insulin producing cell function. Embodiments of the disclosure concern methods of preventing, delaying, or reducing the severity of one or more complications from Type 2 diabetes. Methods encompassed herein also include methods of increasing insulin sensitivity, methods of keeping blood glucose at normal levels, methods of increasing skeletal muscle perfusion, methods of endowing insulin responsiveness, methods of reducing inflammatory mediators, and so forth. In at least some embodiments, the methods and compositions utilized herein are not for Type I diabetes.

Embodiments of the disclosure include methods of increasing insulin sensitivity in a mammal through administering a therapeutically effective amount of a fibroblast cell population and/or fibroblast derivatives. In particular embodiments, the fibroblast cells express the markers CD34 and/or CD133. Although the fibroblasts for any methods herein may originate from any source, in specific embodiments the fibroblast population is derived from a group of tissues selected from the group consisting of cord blood, placenta, skin, bone marrow, amniotic fluid, adipose, umbilical cord matrix, omentum, and subintestinal mucosa; fibroblast derivatives may also be derived therefrom.

In specific cases, the fibroblast cell population possesses an ability to proliferate at a rate of more than one double per 24 hours when cultured at a concentration of 20,000 cells per well in a 96 well plate in 10% fetal calf serum in DMEM media.

In particular embodiments, the fibroblast cell population capable of augmenting insulin sensitivity possesses ability to enhance perfusion of skeletal muscles. The fibroblast cell population is capable of augmenting perfusion of skeletal muscles, in certain embodiments.

Cells capable of increasing insulin sensitivity may be autologous or allogeneic fibroblast expressing the markers CD90, CD105, CD34, CD133 or a combination thereof and/or may substantially lack CD45 and/or CD14 expression. In specific cases, the cell possesses an adherent phenotype and is derived from sources selected from the group consisting of: a) bone marrow; b) peripheral blood; c) endometrium; d) menstrual blood; e) umbilical cord blood; f) deciduous teeth; g) amnion; h) placental matrix; i) muscle tissue; and j) skin.

When providing the fibroblast cells to the individual, the fibroblast cells may be administered intramuscularly, as one example. In specific embodiments, the fibroblast cell population is administered systemically or locally, and in specific cases are administered in proximity to the pancreas.

In any methods herein in which an individual in need of fibroblast cells and/or derivatives thereof, the individual may also receive one or more additional therapeutic agents, such as one or more anti-inflammatory agents.

Embodiments of the disclosure encompass methods of increasing insulin sensitivity in a mammal, in at least some cases through inhibition of one or more inflammatory processes, by administration of a cell population wherein the cells comprise anti-inflammatory activity.

With respect to methods of treating insulin resistance, the insulin resistance may be caused by a number of factors including diabetes, aging, low grade inflammation, obesity, pregnancy, metabolic syndrome X, and congenital abnormality. In one aspect, the disclosure provides cells capable of stimulating perfusion of skeletal muscles, which may be administered systemically or locally into said skeletal muscle with the aim of increasing blood flow.

In one aspect of the disclosure, placental fibroblast cells are utilized, and they may be commercially obtained or isolated from the placental structure and administered for the purpose of increasing perfusion of skeletal muscles. The placental fibroblast cells may be identified based on expression of one or more antigens selected from the group consisting of Oct-4, Rex-1, CD9, CD13, CD29, CD44, CD166, CD90, CD105, SH-3, SH-4, TRA-1-60, TRA-1-81, SSEA-4, Sox-2, and a combination thereof.

In another aspect of the disclosure, bone marrow fibroblasts are utilized and may be commercially obtained or isolated from the bone marrow and administered for the purpose of increasing perfusion of skeletal muscles. The bone marrow fibroblast cells may be generated from bone marrow derived mononuclear cells, said mononuclear cells containing populations capable of differentiating into one or more of the following cell types: endothelial cells, smooth muscle cells, and neuronal cells. In one embodiment, the bone marrow fibroblast cells may be selected based on expression of one or more of the following antigens: CD34, c-kit, flk-1, Stro-1, CD105, CD73, CD31, CD56, CD146, vascular endothelial-cadherin, CD133, CXCR-4, and a combination thereof. Additionally, insulin sensitivity bestowing activity may be enhanced by selecting for cells expressing the marker CD133.

In another aspect of the disclosure, amniotic fluid fibroblast cells may be utilized, such as obtained commercially or isolated from amniotic fluid and used for stimulation of skeletal muscle perfusion and or augmentation of insulin sensitivity. The isolation may be accomplished by purifying mononuclear cells, and/or c-kit expressing cells from amniotic fluid, and the fluid may be extracted by means known to one of skill in the art, including utilization of ultrasound guidance. The amniotic fluid fibroblast cells may be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL, Runx-1, or a combination thereof and/or lack of significant expression of one or more of the following antigens: CD34, CD45, HLA Class II, or a combination thereof.

In another aspect of the disclosure, circulating peripheral blood fibroblast cells are utilized for stimulation of insulin sensitivity. The peripheral blood fibroblast cells may be characterized by the ability to proliferate in vitro for a certain period, such as over 1, 2, 3, 4, 5, 6, or more months, and/or may be characterized by expression of CD34, CXCR4, CD117, CD113, c-met or a combination thereof, and/or characterized by lack of one or more differentiation associated markers. The markers may be selected from one or more of CD2, CD3, CD4, CD11, CD11a, Mac-1, CD14, CD16, CD19, CD24, CD33, CD36, CD38, CD45, CD56, CD64, CD68, CD86, CD66b, HLA-DR or a combination thereof.

In another aspect of the disclosure, tissue fibroblast cells are utilized for stimulation of perfusion of skeletal muscle. The tissue fibroblast cells may express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, THY-1, or a combination thereof. The tissue fibroblast cells may or may not express detectable levels of HLA-DR, CD117, CD45, or a combination thereof. The fibroblast cells may be derived from a group selected from bone marrow, adipose tissue, endometrium, menstrual blood, umbilical cord blood, placental tissue, peripheral blood mononuclear cells, differentiated embryonic stem cells, differentiated progenitor cells, or a combination thereof.

In another aspect of the disclosure, germinal fibroblast cells are utilized for stimulation of perfusion of skeletal muscle, and the cells may express one or more markers selected from the group consisting of Oct4, Nanog, Dppa5 Rbm, cyclin A2, Tex18, Stra8, Dazl, beta1- and alpha6-integrins, Vasa, Fragilis, Nobox, c-Kit, Sca-1, Rex1, and a combination thereof.

In another aspect of the disclosure, adipose tissue-derived fibroblast cells are utilized, for example, for stimulation of perfusion of skeletal muscle, wherein the adipose tissue-derived fibroblast cells may express one or more markers selected from one or more of CD13, CD29, CD44, CD63, CD73, CD90, CD166, Aldehyde dehydrogenase (ALDH), ABCG2, or a combination thereof. In specific embodiments, the adipose tissue derived fibroblast cells may include a population of purified mononuclear cells extracted from adipose tissue capable of proliferating in culture for more than 1, 2, 3, or more months, as an example.

In another aspect of the disclosure, exfoliated teeth-derived fibroblast cells are utilized for stimulation of perfusion of skeletal muscle, wherein the exfoliated teeth-derived fibroblast cells may express one or more markers selected from STRO-1, CD146 (MUC18), alkaline phosphatase, MEPE, bFGF, or a combination thereof.

In another aspect of the disclosure, dermal fibroblast cells are utilized for stimulation of perfusion of skeletal muscle, wherein the cells express one or more markers selected from one or more of CD44, CD13, CD29, CD90, CD105, or a combination thereof and may be capable of proliferating in culture for a period of at least 1, 2, 3, or more months.

In another aspect of the disclosure, side population fibroblasts cells (a term known in the art and such as may be identified based on expression of multidrug resistance transport protein (ABCG2), for example, and/or an ability to efflux intracellular dyes, such as rhodamine-123 and/or Hoechst 33342) are utilized for stimulation of insulin sensitivity and perfusion of skeletal muscle, and the side population cells may be identified based on expression of multidrug resistance transport protein (ABCG2), for example, and/or an ability to efflux intracellular dyes such as rhodamine-123 and/or Hoechst 33342. Side population cells may be derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue, as examples.

In another aspect of the disclosure, fibroblast progenitor cells are utilized for stimulation of perfusion of skeletal muscle. In one aspect, fibroblast progenitors are collected from mobilized peripheral blood. The mobilization may be accomplished by administration of one or more mobilizing agents or therapies. The mobilizing agent(s) may be selected from the group consisting of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA)reductase inhibitors, small molecule antagonists of SDF-1, and a combination thereof. The mobilization therapy may be selected from one or more of exercise, hyperbaric oxygen, autohemotherapy by ex vivo ozonation of peripheral blood, and/or induction of SDF-1 secretion in an anatomical area outside of the bone marrow. In some aspects of the disclosure, fibroblast progenitor cells express one or more markers such as CD31, CD34, AC133, CD146 and/or flk1.

In one aspect of the disclosure, cells encompassed herein as fibroblasts or fibroblast progenitors may be administered systemically or in proximity to one or more particular tissues and/or organs in order to provide cellular and/or trophic support for regeneration of insulin producing cells. In specific embodiments, they are provided in proximity to the pancreas. They may be also administered intravenously, intramuscularly, intraperitoneally, and intralymphatically.

In one aspect of the disclosure, one or more anti-inflammatory agents may be administered to an individual receiving the fibroblast cells that increase skeletal muscle perfusion, and/or for regeneration of insulin-producing cells. The anti-inflammatory agent(s) may inhibit molecular pathways such as the NF-kappa B pathway, the MyD88 pathway, the TNF signal transduction pathway, the Toll like receptor signal transduction pathway pathways associated with upregulation of MHC expression, upregulation of C-reactive protein production, and/or upregulation of TNF alpha production. Anti-inflammatory agents useful for the methods of the disclosure include at least Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; Zomepirac Sodium, IL-4, IL-10, IL-13, IL-20, IL-1 receptor antagonist, TGF-beta, and a combination thereof.

The current disclosure encompasses insulin sensitivity that can be increased through augmentation of muscular perfusion, and/or decreasing inflammation, and/or providing means for islet regeneration. The disclosure provides means of treating a variety of conditions associated with insulin resistance outside of IDDM and NIDDM, including conditions such as gestational diabetes; carbohydrate and lipid metabolism abnormalities; glucosuria; micro- and macrovascular disease; polyneuropathy and diabetic retinopathy; diabetic nephropathy; insulin resistance; impaired glucose tolerance (or glucose intolerance); obesity; hyperglycemia (elevated blood glucose concentration); hyperinsulinemia; hyperlipidemia; hyperlipoproteinemia; atherosclerosis; hypertension; congenital or acquired digestion and absorption disorder including malabsorption syndrome; disease caused by loss of a mucosal barrier function of the gut; and/or protein-losing gastroenteropathy.

Other conditions associated with above-normal blood glucose concentration either in an acute or chronic form are also encompassed by the disclosure. Thus, embodiments of the disclosure include methods of reducing blood glucose concentration by administering to an individual with elevated blood glucose concentration a therapeutically effective amount of fibroblasts and/or fibroblast derivatives. In one embodiment elevated glucose levels are fasting levels higher than 100 mg/dL.

The disclosure encompasses ways of utilizing cells, and in specific aspects, fibroblasts with ability to differentiate into other cells, for the purposes of stimulating muscle perfusion, decreasing inflammatory mediator production, and in some situations allowing for pancreatic regeneration. Also provided are means of inducing islet regeneration in an environment conducive to maintenance of viability and function of the islets or components thereof.

In one aspect of the disclosure, increasing angiogenic potential of a subject is performed with the purpose of increasing vascularity of the pancreas and may include delivery of fibroblasts and/or derivatives thereof. The increase in angiogenic potential may be performed through administration of one or more angiogenic factors, cells with angiogenic ability, or a combination thereof. Angiogenesis may be stimulated in the context of anti-inflammatory intervention with or without administration of cells capable of differentiating into insulin-producing cells.

In one aspect of the disclosure, an individual is treated with one or more agents known to stimulate generation of endogenous insulin-producing cells, while at the same time increasing anti-inflammatory and/or angiogenic activity. Methods are known in the art for increasing endogenous insulin-producing cell differentiation. One example of such a method is administration of a combination EGF and gastrin, which has been demonstrated to induce insulin secretion through differentiation of endogenous stem cells into insulin-producing cells [12-14].

In one aspect of the disclosure, one or more anti-inflammatory agents are used together with fibroblast cells and/or derivatives thereof capable of increasing angiogenesis and/or inducing islet neogenesis.

In one embodiment of the disclosure, patients suffering from insulin resistance, having a state of NIDDM are treated by intramuscular administration of fibroblast cells and/or derivatives thereof. It is known that 70-80% of post-prandial glucose is metabolized by skeletal muscle [15]. In many patients with NIDDM, profound atherosclerotic deposits are known to inhibit circulation of the extremities. Without being bound to theory, inhibition of circulation may be occurring at vessels such as the femoral artery, the popliteal artery and/or the tibial arteries. Additionally inhibition of circulation may be occurring at the level of capillaries feeding various muscles. Impaired circulation is known to occur not only due to atherosclerosis, but also due to inhibited vasodilatory mechanisms [16]. Because of inhibited circulation and vasodilatory responses, insulin activation of GLUT4 membrane localization and general insulin responsiveness is blunted. Accordingly in one embodiment of the current disclosure, the ability of muscles to respond to insulin is improved by administration of fibroblast cells capable of restoring endothelial function, as well as inducing angiogenesis. The fibroblast cells useful for this purpose may be of autologous, endogenous, or allogeneic origin.

In one particular embodiment an individual with NIDDM is treated with fibroblasts by administration approximately 1.5 cm deep into the gastrocnemius muscle. Injections may be performed to deliver a total number of fibroblast cells ranging from 10 million to 10 billion mononuclear cells. In a preferred embodiment injections of approximately 1-3 billion mononuclear cells are administered. The injections may be performed with a total injection volume of 10-50 ml, with injections being distributed on a grid placed on the gastrocnemius muscle. Number of injections may range from 1-100 injections, with an optimum number ranging approximately from 10-50 injections, and more optimally between 20-30 injections. Injection of fibroblast mononuclear cells may be performed specifically in an area of occlusion identified by methods known in the art, such as digital subtractive angiography, Doppler imaging, positron emission tomography, and ultrasound. Alternatively, administration of fibroblast cells may be performed in areas in which occlusion is suspected by not established. Additionally, means of assessing tissue oxygenation such as transcutaneous pulse oximetry may be used to identify muscular areas deficient in oxygenation. Deficiencies in general circulation may also be identified by measurements such as toe pulse, or by the ankle-brachial index. In one embodiment administration of bone marrow fibroblast cells is performed in the gastrocnemious muscle is the ankle brachial index is below 0.9. In other embodiments, administration of fibroblast cells is performed in various muscles regardless of perfusion status. For example, patients with NIDDM may be injected with numerous aliquots of bone marrow in major skeletal muscles. Examples of major skeletal muscles suitable for injection include: the deltoid, pectoralis major, biceps, rectus abdominus, external oblique, gluteus medius, gluteus maximus, soleus, tibialis anterior, vastus medialis, vastus intermedius, vastus lateralis, rectus femoris, and the sartorious muscles.

In embodiments wherein fibroblasts are utilized to increase muscular perfusion, including at least skeletal muscular perfusion, the effects may or may not be monitored. The effects of intramuscular fibroblast cell administration, as one example, may be observed not only by an ability to increase perfusion, but also an ability to augment the flow-mediated dilation response (any vasodilatation of an artery following an increase in luminal blood flow and internal-wall shear stress). In specific embodiments, the effect of cell administration is assessed by various means known in the art for quantification of insulin sensitivity. For example, the hyperinsulinemic-euglycemic clamp technique is considered a golden standard for this purpose, however because of impracticalities such as time and expense, other techniques may also be used. Such techniques include the frequently sampled IV glucose tolerance test (FSIVGTT), insulin tolerance test (ITT), insulin sensitivity test (IST), the continuous infusion of glucose with model assessment (CIGMA) and the oral glucose tolerance test (OGTT).

Treatment with bone marrow mononuclear cells may be performed, in some embodiments of the invention, in conjunction with cytokines known to mobilize endogenous fibroblast cells. It is known that intramuscular administration of bone marrow mononuclear cells causes systemic mobilization of endogenous CD34 fibroblast cells from the bone marrow [17]. Accordingly, the current disclosure encompasses methods wherein subsequent to administration of bone marrow mononuclear cells into muscle of a patient with NIDDM, augmentation of endogenous fibroblast cell mobilization will evoke an enhanced therapeutic effect. Because the intramuscularly administered fibroblast cells possess chemotactic activity, the mobilization of bone marrow fibroblast cells through administration of factors such as G-CSF, GM-CSF, M-CSF, and/or moxibil, will augment a therapeutic effect. Administration of G-CSF may be performed concurrently with intramuscular injection of bone marrow cells, or may be performed near the time point associated with maximal mobilization of CD34 cells. The timepoint may be determined experimentally, or may be based on previously published data. It is reported, for example, that maximal CD34 mobilization subsequent to administration of bone marrow cells intramuscularly occurs around day 30. Accordingly, in one embodiment of methods of the disclosure, G-CSF is administered prior to day 30, at concentrations sufficient to evoke endogenous CD34 mobilization. In one embodiment, G-CSF is administered at a concentration of approximately 60 micrograms per day be subcutaneous injection for 5 days. Administration may be performed, for example, starting on day 25 subsequent to intramuscular injection of bone marrow cells. In some embodiments, one can concurrently administer heparin so as to avoid the possibility of causing embolism because of high systemic leukocyte counts caused by the G-CSF injection. This is useful in patients with NIDDM who are already at a higher risk of embolisms in comparison to the general population. Anticoagulation methods are well known in the art and may utilize agents besides heparin. However, if heparin anticoagulation is used, then approximate doses of 10,000 units per day may be useful, as one example.

In another embodiment, fibroblast cells are administered as encompassed herein in combination with one or more agents known to increase regenerative activity. Such agents may include, for example, erythropoietin [18], prolactin [19], human chorionic gonadotropin (as described in U.S. Pat. No. 5,968,513 and incorporated by reference), gastrin [20], EGF [12], FGF [21], and/or VEGF [22]. In one embodiment, administration of neutralizers of TNF alpha are concurrently administered with fibroblasts to de-repress inhibitory effects of this cytokine on circulating fibroblasts cells [23].

### III. Fibroblasts and Modifications and Preparations Thereof

Fibroblasts utilized in methods of the disclosure may be prepared and provided to an individual in need thereof. The fibroblasts may be autologous or allogeneic or xenogeneic with respect to the individual being treated.

In particular embodiments, fibroblasts that have the ability to augment perfusion, to increase insulin sensitivity, to treat insulin resistance, to provide an environment suitable for restoration of insulin-producing cell function and so forth are modified and/or prepared for use in methods of the disclosure. The fibroblasts of the disclosure may or may not have a particular expression profile. In some embodiments, the fibroblasts of the disclosure express one or more particular markers. In specific embodiments, the fibroblasts express CD34, CD133, or both. In particular embodiments, the fibroblasts express one or more markers selected from the group consisting of CD34, c-kit, flk-1, Stro-1, CD105, CD73, CD31, CD56, CD146, vascular endothelial-cadherin, CD133, CXCR-4, and a combination thereof. In specific embodiments, the fibroblasts express CD34, CD133, and 1, 2, 3, 4, 5, 6, 7, 8, 9, or all of c-kit, flk-1, Stro-1, CD105, CD73, CD31, CD56, CD146, vascular endothelial-cadherin, and CXCR-4. In specific embodiments, the fibroblast cells are CD34+, CD133+, or both. In certain embodiments, the fibroblasts are CD90+, CD105+, and substantially lack CD45 and/or CD14 expression.

In specific embodiments, the fibroblasts have regenerative activity.

In specific embodiments, the fibroblasts are present in a culture, whether for storage and/or preparation. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more sub-cultivations of a primary cell culture. Each round of sub-culturing is referred to as a passage. When cells are sub-cultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

In some embodiments, the fibroblasts are comprised in a conditioned medium or have been in or exposed to a conditioned medium. A conditioned medium is a medium in which a specific cell or population of cells has been cultured, and then removed. When cells are cultured in a medium, they may secrete one or more cellular factors that can provide trophic support to other cells or have another function. Generally, a trophic factor is defined as a substance that promotes or at least supports, survival, growth, proliferation and/or maturation of a cell, or stimulates increased activity of a cell. Such trophic factors include, but are not limited to hormones, cytokines, extracellular matrix (ECM), proteins, vesicles, antibodies, and granules. The medium containing the cellular factors is the conditioned medium. The fibroblasts may secrete one or more factors or entities (Such as exosomes) that are utilized for a medical purpose either alone or in conjunction with one or more other components.

As used herein, the term Growth Medium generally refers to a medium sufficient for the culturing of fibroblast cells of any kind. In particular, one medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotic s/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present disclosure, the term standard growth conditions, as used herein refers to culturing of cells at 37° C., in a standard atmosphere comprising 5% CO₂. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

The cells may be prepared for administration in a pharmaceutically acceptable carrier, for example a sterile saline isotonic solution. In some embodiments, the pharmaceutically acceptable carrier may comprise one or more additional agents, such as FAS ligand, IL-2R, IL-1 Ra, IL-2, IL-4, IL-8, IL-10, IL-20, IL-35, HLA-G, PD-L1, I-309, IDO, iNOS, CD200, Galectin 3, sCR1, arginase, PGE-2, aspirin, atorvastatin, fluvastatin, lovastatin, pravastatin, rosuvastatin, simvastatin, pitavastatin, n-acetylcysteine, rapamycin, IVIG, naltrexone, TGF-beta, VEGF, PDGF, CTLA-4, anti-CD45RB antibody, hydroxychloroquine, leflunomide, auranofin, dicyanogold, sulfasalazine, methotrexate, glucocorticoids, etanercept, adalimumab, abatacept, anakinra, certolizumab, Etanercept-szzs, golimumab, infliximab, rituximab, tocilizumab, cyclosporine, IFN-gamma, everolimus, rapamycin, VEGF, FGF-1, FGF-2, angiopoietin, HIF-1-alpha, or a combination thereof.

In one embodiment of the disclosure, fibroblasts are administered to a subject by any suitable route, including by injection (such as intramuscular injection), including in hypoxic areas. Suitable routes include intravenous, subcutaneous, intrathecal, oral, intrarectal, intrathecal, intra-omentral, intraventricular, intrahepatic, and intrarenal.

In certain embodiments, fibroblasts may be derived from tissues comprising skin, heart, blood vessels, bone marrow, skeletal muscle, liver, pancreas, brain, adipose tissue, foreskin, placental, and/or umbilical cord. In specific embodiments, the fibroblasts are placental, fetal, neonatal or adult or mixtures thereof.

The number of administrations of cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

In some embodiments, the cells are subjected to one or more media compositions that comprises, consists of, or consists essentially of Roswell Park Memorial Institute (RPMI-1640), Dublecco's Modified Essential Media (DMEM), Eagle's Modified Essential Media (EMEM), Optimem, Iscove's Media, or a combination thereof.

In one embodiment of the disclosure, fibroblasts cells are collected from amniotic fluid or amniotic membrane. The amniotic derived fibroblast cells may be utilized therapeutically in an unpurified manner optionally subsequent to matching. The amniotic fibroblast cells may be administered locally, intramuscularly or systemically in a patient suffering from insulin resistance. In other embodiments, amniotic fibroblast cells are substantially purified based on expression of markers such as SSEA-3, SSEA4, Tra-1-60, Tra-1-81 and Tra-2-54, and subsequently administered. In other embodiments cells are cultured, for example as described in US Patent Application Publication No. US 2005/0054093, expanded, and subsequently infused into the patient Amniotic fibroblast cells are described in the following references [24-26]. One particular aspect of amniotic fibroblast cells that makes them amenable for use in practicing certain aspects of the current disclosure is their bi-phenotypic profile as being both mesenchymal and endothelial progenitors; this allows for anti-inflammatory, as well as angiogenic function [25, 27]. This property is useful for treatment of patients with insulin resistance and associated diseases that would benefit from angiogenesis, but also from the anti-inflammatory effects of fibroblast cells. The use of amniotic fluid fibroblast cells is particularly useful in situations such as ischemia-associated pathologies and/or inflammatory states, in which hypoxia is known to perpetuate degenerative processes.

In one embodiment, allogeneic or autologous donors that have been matched with HLA or mixed lymphocyte reaction are mobilized by administration of G-CSF (filgrastim: neupogen) at a concentration of approximately 10 ug/kg/day by subcutaneous injection for 2-7 days, such as 4-5 days. Peripheral blood mononuclear cells are collected using an apheresis device such as the AS104 cell separator (Fresenius Medical). 1-40×10⁹ mononuclear cells are collected, concentrated and administered locally, injected systemically, or in an area proximal to the region pathology associated with the given degenerative disease. In situations where ischemia is localized cellular administration may be performed within the context of the current invention. Methods of identification of such areas of ischemia is routinely known in the art and includes the use of techniques such as nuclear or MRI imagining. Variations of this procedure may include steps such as subsequent culture of cells to enrich for various populations known to possess angiogenic and/or anti-inflammatory, and/or anti-remodeling, and/or regenerative properties. Additionally cells may be purified for specific subtypes before and/or after culture. Treatments can be made to the cells during culture or at specific time points during ex vivo culture but before infusion in order to generate and/or expand specific subtypes and/or functional properties. The various embodiments of the invention for other fibroblast cells described in this disclosure can also be applied for circulating peripheral blood fibroblast cells.

In one embodiment of the disclosure, allogeneic or autologous adipose tissue-derived fibroblast cells are used as a cell source. The adipose tissue derived fibroblast cells express markers such as CD9; CD29 (integrin beta 1); CD44 (hyaluronate receptor); CD49d,e (integrin alpha 4, 5); CD55 (decay accelerating factor); CD105 (endoglin); CD106 (VCAM-1); CD166 (ALCAM). These markers are useful not only for identification but may be used as a means of positive selection, before and/or after culture in order to increase purity of the desired cell population. In terms of purification and isolation, devices are known to those skilled in the art for rapid extraction and purification of cells adipose tissues. U.S. Pat. No. 6,316,247 describes a device that purifies mononuclear adipose-derived fibroblast cells in an enclosed environment without the need for setting up a GMP/GTP cell processing laboratory so that patients may be treated in a wide variety of settings. One embodiment of the disclosure involves attaining 10-200 ml of raw lipoaspirate, washing said lipoaspirate in phosphate buffered saline, digesting said lipoaspirate with 0.075% collagenase type I for 30-60 min at 37° C. with gentle agitation, neutralizing said collagenase with DMEM or other medium containing autologous serum, preferably at a concentration of 10% v/v, centrifuging the treated lipoaspirate at approximately 700-2000 g for 5-15 minutes, followed by resuspension of said cells in an appropriate medium such as DMEM. Cells are subsequently filtered using a cell strainer, for example a 100 μm nylon cell strainer in order to remove debris. Filtered cells are subsequently centrifuged again at approximately 700-2000 g for 5-15 minutes and re-suspended at a concentration of approximately 1×10⁶/cm² into culture flasks or similar vessels. After 10-20 hours of culture non-adherent cells are removed by washing with PBS and remaining cells are cultured at similar conditions as described for culture of cord blood derived fibroblast cells. Upon reaching a concentration desired for clinical use, cells are harvested, assessed for purity and administered in a patient in need thereof as described above.

Unique, tissue-specific fibroblast cells may be used in the autologous or allogeneic setting for the practice of the methods of the disclosure. These cells may be used whole, or induced to differentiate into endothelial or endothelial precursor cells. Cells expressing the ability to efflux certain dyes, including but not limited to rhodamin-123, are associated with stem cell-like properties [28] and may be utilized The cells can be purified from tissue subsequent to cell dissociation, based on efflux properties. Accordingly, in one embodiment of the current disclosure, tissue-derived side population cells used in conjunction with fibroblasts may be utilized either freshly isolated, sorted into subpopulations, or subsequent to ex vivo culture, for the treatment of degenerative conditions. For use in the disclosure, side population cells may be derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue. Purification of side population cells can be performed, in one embodiment, by re-suspending dissociated cardiac valve cells at 10⁶ cells/ml, and staining with 6.0 μg/ml of Hoechst 33342 in calcium- and magnesium-free HBSS+(supplemented with 2% FCS, 10 mM Hepes, and 1% penicillin/streptomycin) medium for 90 min at 37° C. Cells are then run on a flow cytometer and assessed for efflux of Hoechst 33342. Purified cells may be assessed for ability to form cardiac spheres, this may be performed by suspending said side population cells at a density of 1-2×106 cells/ml in 10-cm uncoated dishes in DME/M199 (1:1) serum-free growth medium containing insulin (25 μg/ml), transferin (100 μg/ml), progesterone (20 nM), sodium selenate (30 nM), putrescine (60 nM), recombinant murine EGF (20 ng/ml), and recombinant human FGF2. Half of the medium is changed every 3 d. Passaging may be performed using 0.05% trypsin and 0.53 mM EDTA-4Na every 7-14 d. Cardiospheres are then dissociated into a single-cell suspension then used either for therapeutic purposes, or for evaluating therapeutic ability in vitro or in animal models before clinical use. The cardiospheres can be induced to differentiate into endothelial cells by culture in angiogenic factors prior to administration. These methods have been described for side population cells of other tissues in publications to which the practitioner of the methods of the disclosure is referred to [29-31]. The various embodiments of the invention for other fibroblast cells described in this disclosure can also be applied for side population fibroblast cells.

In one embodiment of the disclosure, “young” fibroblast cells are used to compensate for deteriorating function of senescent tissue. The term “young” is used to denote cells derived from a donor of an age younger than the recipient. In some embodiments, young cells may be cells of the same recipient that were collected at an earlier date to infusion of cells. There are certain advantages for utilization of young cells for the practice of the current disclosed methods. For example, it is known that aged animals possess impaired physiological responses in comparison to younger animals. Aging is known to be associated with impaired insulin responsiveness [32, 33]. In some cases senescence is associated with increased production of inflammatory cytokines such as TNF-alpha, which cause insulin resistance. For example, it was demonstrated that antibodies to TNF-alpha are capable of inhibiting age-related insulin resistance of muscles of Sprague-Dawley rats [34]. For example, in an experiment by Edelberg's group, it was demonstrated that 3 month old ROSA beta galactosidase transgenic bone marrow cells, when transferred into an 18-month old recipient are capable of entering the bone marrow and causing chimeric hematopoiesis in absence of recipient conditioning [35]. More interestingly, it was demonstrated that endothelial progenitor cells from the young 3 month old bone marrow donor are capable of “rejuvenating” 18 month old recipient mouse ability to sustain vascularization of neonatal hearts transplanted ectopically. Specifically, when 18 month old recipients were transplanted with neonatal hearts, donor hearts lost viability due to lack of vascularization. If 18 month old bone marrow cells were administered into the 18 month old recipient, ability to vascularize the neonatal heart was still impaired. However, 3 month old bone marrow infusion was capable of establishing vascularization in a dose-dependent and PDGF-B dependent manner.

In one embodiment of the disclosure, fibroblast cells, substantially younger than a recipient are administered into said recipient for production of cells that directly or indirectly increase responsiveness to insulin. As previously stated, fibroblast cells derived from cord blood, bone marrow, and adipose tissue are capable of differentiating into skeletal muscle. Taking the observation that younger cells are capable of integrating with older tissue and re-establishing function of older tissue, the invention teachings the use of younger fibroblast cells for increasing responsiveness to insulin. In one embodiment cord blood fibroblast cells are utilized as a source of “young” fibroblast cells for generation of cells similar to skeletal muscle cells in vivo in order to decrease insulin resistance. This is not to be interpreted as being bound to theory since the differentiation into muscle-like cells is one of several mechanisms by which the invention discloses ability of cord blood fibroblast cells to reverse insulin resistance. In one embodiment, the cord blood fibroblast cells are obtain from a cord blood sample obtained from a healthy pregnancy. Umbilical cord blood is purified according to routine methods [36]. In one embodiment, a 16-gauge needle from a standard Baxter 450-ml blood donor set containing CPD A anticoagulant (citrate/phosphate/dextrose/adenine) (Baxter Health Care, Deerfield, Ill.) is inserted and used to puncture the umbilical vein of a placenta obtained from a mother tested for viral and bacterial infections according to international donor standards. Cord blood is allowed to drain by gravity so as to drip into the blood bag. The placenta is placed in a plastic-lined, absorbent cotton pad suspended from a specially constructed support frame in order to allow collection and reduce the contamination with maternal blood and other secretions, The 63 ml of CPD A used in the standard blood transfusion bag, calculated for 450 ml of blood, is reduced to 23 ml by draining 40 ml into a graduated cylinder just prior to collection. An aliquot of the cord blood is removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch is added to the anticoagulated cord blood to a final concentration of 1.2%. The leukocyte rich supernatant is then separated by centrifuging the cord blood hydroxyethyl starch mixture in the original collection blood bag (50×g for 5 min at 10° C.). The leukocyte-rich supernatant is transferred from the bag into a 150-ml Plasma Transfer bag (Baxter Health Care) and centrifuged (400×g for 10 min) to sediment the cells. Surplus supernatant plasma is transferred into a second plasma transfer bag without severing the connecting tube. Finally, the sedimented leukocytes are re-suspended in supernatant plasma to a total volume of 20 ml. Approximately 5×10⁸-7×10⁹ nucleated cells are obtained per cord. Cells are cryopreserved according to the method described by Rubinstein et al [36].

In some embodiments, matching of donor cells to recipient is performed, in other situations it is not. As one example, a group of multiple cord blood fibroblast cell sources, purified and cryopreserved as described above, may be utilized for treatment of a patient in need of fibroblast cell therapy. An aliquot of mononuclear cells from each of the multiple cord blood fibroblast cell source is taken, said aliquot comprising approximately 10⁵ cells. The cells are plated in Nunc 96-well plates at a concentration of 10⁴ cells per well in 9 wells in a volume of 100 uL per well. Prior to plating, the cells are washed and reconstituted in DMEM-LG media (Life Technologies), supplemented with 10% heat-inactivated fetal calf serum. Said cord blood cells are considered “stimulators” for the purpose of the matching procedure. In order to generate “responder” cells, 20 ml of peripheral blood is extracted from the patient in need of fibroblast cell therapy through venipuncture. The 20 ml of peripheral blood is heparinized by drawing in a heparinized Vacutainer™, is layered on Ficoll™ density gradient and centrifuged for approximately 60 minutes at 500 g. The mononuclear layer is harvested and washed in phosphate buffered saline supplemented with 3% fetal calf serum. For every 9 wells of stimulator cells, to 3 wells, a concentration of 10⁴ responder cells are added, to 3 wells a concentration of 10⁵ responder cells are added, and to 3 wells, media with no cells are added in order to have a control for spontaneous activity of stimulator cells. Responder cells are reconstituted in DMEM-LG media, supplemented with 10% heat-inactivated fetal calf serum before being added to stimulator cells. Responder cells and media comprise a volume of 100 uL before being added to stimulator cells. Additionally, in order to have a control for spontaneous activity of responder cells, 10⁴ and 10⁵ responder cells in a volume of 100 uL are added in triplicate to 100 uL of media without stimulator cells. To have a control for background or other contaminations, 3 wells are plated with 200 uL of media alone. Accordingly, the total culture consists of 25 fibroblast cell sources×9 wells=225 wells, that is, a total of three 96-well plates are used. Additionally, 9 wells are used for the responder controls in which no stimulator cells, or no cells at all are added. Seventy-two-hour mixed lymphocyte reaction is performed and the cells were pulsed with 1 μCi [3H]thymidine for the last 18 h. The cultures are harvested onto glass fiber filters (Wallac, Turku, Finland). Radioactivity is counted using a Wallac 1450 Microbeta liquid scintillation counter and the data were analyzed with UltraTerm 3 software (Microsoft, Seattle, Wash.). If lymphocyte proliferation is more than 2 fold higher as compared to lymphocytes cultured without stimulator cells, when subtracting the background proliferation of stimulators alone, then the cord blood batch is not used for therapy. According to this criteria, 2 or more of the multiple batches of fibroblast cell sources may be chosen for administration into the patient. Cells purified may be utilized for delivery.

In one embodiment, cord blood is used to obtain fibroblasts with or without matching to the recipient, however steps may be taken so as to deplete the cord blood of specific immunogenic components that may cause host versus graft, and/or alternatively, the graft is manipulated so as to neutralize immunological cells that may have the potential to cause graft versus host. Specifically, cord blood fibroblast cells are concentrated in Good Manufacturing Practices (GMP) grade-Hanks balanced salt solution (comprising Ca²⁺). Cells are washed previously to concentration so that said cells are substantially free from plasma and depleted of red blood cells and granulocytes. The volume of the mononuclear cell suspension is adjusted so that the cell density is approximately 3×10⁷/mL, and CAMPATH-1M or CAMPATH-1H is added to give a final concentration of 0.1 mg/mL. The mixture is incubated for 15 minutes at room temperature, and then recipient serum is added to achieve final concentration of 25% (vol/vol). The mixture is subsequently incubated for a further 30 minutes at 37° C. The treated cord blood cells are washed once, assessed for viability, and infused into a patient in need of therapy.

The ability of fibroblast cells to differentiate into various tissues is well known, however, a lesser known ability of various fibroblast cells is their anti-inflammatory function. It is established that NIDDM is associated with elevation of inflammatory mediators. This was elegantly overviewed in a review by Pickup et al. who described a “low grade inflammation” as part of the process associated with development of insulin resistance and subsequent NIDDM. This is based on observations that elevated circulating inflammatory markers such as C-reactive protein and interleukin-6 predict the development of type 2 diabetes, and several drugs with anti-inflammatory properties lower both acute-phase reactants and glycemia (aspirin and thiazolidinediones) and possibly decrease the risk of developing type 2 diabetes (statins). Additionally Pickup postulates that features of type 2 diabetes, such as fatigue, sleep disturbance, and depression may be the result of syfibroblastic “hypercytokinemia” [37]. It is known that TNF-alpha and IL-6 are secreted at a basal level by the adipose compartment and correlations have been made between levels of these cytokines and resistance to insulin. For example, Kern et al measured TNF and IL-6 levels in non-diabetic lean and obese patients. When lean [body mass index (BMI)<25 kg/m(2)] and obese (BMI 30-40 kg/m(2)) subjects were compared, there was a 7.5-fold increase in TNF secretion, and the TNF secretion was inversely related to insulin sensitivity as measured by the intravenous glucose tolerance test [38]. Numerous other studies have demonstrated high levels of TNF in plasma of patients that are insulin resistant [39, 40]. Additionally, reduction in TNF-alpha is associated with response to various insulin sensitizers [41]. The ability of TNF-alpha to induce insulin resistance is believed to be based on induction of serine phosphorylation of insulin receptor substrate-1 (IRS-1). IRS-1 serine phosphorylation causes dissociation of IRS proteins from the insulin receptor, thus blocking insulin signal transduction [42]. Despite the important role of TNF-alpha in insulin resistance, it is not the only causative factor. Treatment with TNF-alpha blocking agents appears not to increase insulin sensitivity [43, 44]. This, however, is most likely due to the plethora of inflammatory agents such as leptin, IL-6, resistin, visfatin and IL-1 that are secreted by adipose tissue and associated with insulin resistance in addition to TNF-alpha [45, 46]. In rheumatoid arthritis TNF-alpha is one of the major cytokines produced, and as a result insulin resistance develops. Interestingly, blockade of TNF-alpha using infliximab in RA patients results in increased insulin sensitivity [47]. This finding may be explained by the fact that RA is associated with one major inflammatory mediator, whereas obesity is associated with several. Accordingly, in one embodiment of the disclosure, fibroblast cells are used to induce an anti-inflammatory state or to reduce inflammation in a patient with NIDDM. The inflammatory state may be diagnosed by many means available to one of skill in the art, including assessment of C-reactive protein levels, IL-1, IL-6, TNF, leptin, and IL-18. Various fibroblast cell sources may be used in the practice of the invention. Additionally, the combination of fibroblast cell for the generation of angiogenesis, together with fibroblast cells for the induction of an anti-inflammatory state is disclosed in the current disclosure. The cells that are useful may include, in some embodiments, fibroblast cells. These cells have been shown to possess immune suppressive and anti-inflammatory functions.

In some embodiments of the disclosure, fibroblast cell populations are used together with one or more agents known to stimulate production of insulin or protect islets from damage. For example, such agents may be amylin analogs. These compounds duplicate the effect of amylin by delaying gastric emptying, decreasing postprandial glucagon release, and modulating appetite. Pramlintide acetate, sold under the name Symlin is indicated as an adjunct to mealtime insulin for the treatment of patients with type 1 and type 2 diabetes. In numerous clinical trials, adjunctive pramlintide treatment resulted in improved postprandial glucose control and significantly reduced A1C and body weight compared with insulin alone. Numerous patents have been issued for various agents capable of stimulating insulin secretion and/or sensitizing peripheral tissue to insulin activity. These include, for example, U.S. Pat. Nos. 6,121,282, 6,057,343, 6,048,842, 6,037,359, 6,030,990, 5,990,139, 5,981,510, 5,980,902, 5,955,481, 5,929,055, 5,925,656, 5,925,647, 5,916,555, 5,900,240, 5,885,980, 5,849,989, 5,837,255, 5,830,873, 5,830,434, 5,817,634, 5,783,556, 5,756,513, 5,753,790, 5,747,527, 5,731,292, 5,728,720, 5,708,012, 5,691,386, 5,681,958, 5,677,342, 5,674,900, 5,545,672, 5,532,256, 5,531,991, 5,510,360, 5,480,896, 5,468,762, 5,444,086, 5,424,406, 5,420,146, RE34,878, 5,294,708, 5,268,373, 5,258,382, 5,019,580, 4,968,707, 4,845,231, 4,845,094, 4,816,484, 4,812,471, 4,740,521, 4,716,163, 4,695,634, 4,681,898, 4,622,406, 4,499,279, 4,467,681, 4,448,971, 4,430,337, 4,421,752, 4,419,353, 4,405,625, 4,374,148, 4,336,391, 4,336,379, 4,305,955, 4,262,018, 4,220,650, 4,207,330, 4,195,094, 4,172,835, 4,164,573, 4,163,745, 4,141,898, 4,129,567, 4,093,616, 4,073,910, 4,052,507, 4,044,015, 4,042,583, 4,008,245, 3,992,388, 3,987,172, 3,961,065, 3,954,784, 3,950,518, 3,933,830, which are incorporated herein by reference in their entirety.

### IV. Kits of the Disclosure

Certain aspects of the present disclosure also concern kits containing compositions of the disclosure or compositions to implement methods of the disclosure. In some embodiments, kits can be used to provide fibroblasts, including fibroblast regenerative cells, populations thereof, progeny thereof or conditioned media thereof. In some cases, kits include one or more reagents for producing and/or identifying fibroblast cells, including regenerative cells.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

In certain aspects, negative and/or positive control agents are included in some kit embodiments. The control molecules can be used to verify the enhance regenerative activity of fibroblast cells.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

The kit may or may not comprise one or more additional therapies for the medical condition being treated, including for diabetes such as metformin, one or more sulfonylureas, one or more meglitinides, one or more thiazolidinediones, one or more DPP-4 inhibitors, one or more GLP-1 receptor agonists, one or more SGLT2 inhibitors, and/or insulin. The kit may or may not comprise one or more devices and/or reagents for diagnosis of diabetes and/or monitoring of blood sugar level.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Increased Insulin Responsiveness in Type 2 Diabetes

A group of 100 patients are recruited with type 2 diabetes receiving daily insulin injections. 50 patients are treated with placebo control and 50 receive allogeneic cord blood derived fibroblast cells. Cells are injected intramuscularly in the gastrocnemius muscle as described in the literature (Durdu et al. J Vasc Surg. 2006 October; 44(4):732-9) with a concentration of 40 million cells per limb. Cord blood CD34 extraction and expansion are described below. Umbilical cord blood is purified according to routine methods ((Rubinstein, et al. Processing and cryopreservation of placental/umbilical cord blood for unrelated bone marrow reconstitution. Proc Natl Acad Sci USA 92:10119-10122). Briefly, a 16-gauge needle from a standard Baxter 450-ml blood donor set containing CPD A anticoagulant (citrate/phosphate/dextrose/adenine) (Baxter Health Care, Deerfield, Ill.) is inserted and used to puncture the umbilical vein of a placenta obtained from healthy delivery from a mother tested for viral and bacterial infections according to international donor standards. Cord blood is allowed to drain by gravity so as to drip into the blood bag. The placenta is placed in a plastic-lined, absorbent cotton pad suspended from a specially constructed support frame in order to allow collection and reduce the contamination with maternal blood and other secretions, The 63 ml of CPD A used in the standard blood transfusion bag, calculated for 450 ml of blood, is reduced to 23 ml by draining 40 ml into a graduated cylinder just prior to collection. This volume of anticoagulant matches better the cord volumes usually retrieved (<170 ml).

An aliquot of the blood is removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch is added to the anticoagulated cord blood to a final concentration of 1.2%. The leukocyte rich supernatant is then separated by centrifuging the cord blood hydroxyethyl starch mixture in the original collection blood bag (50×g for 5 min at 10° C.). The leukocyte-rich supernatant is expressed from the bag into a 150-ml Plasma Transfer bag (Baxter Health Care) and centrifuged (400×g for 10 min) to sediment the cells. Surplus supernatant plasma is transferred into a second plasma Transfer bag without severing the connecting tube. Finally, the sedimented leukocytes are resuspended in supernatant plasma to a total volume of 20 ml. Approximately 5×10⁸-7×10⁹ nucleated cells are obtained per cord. Cells are cryopreserved according to the method described by Rubinstein et al (Rubinstein, et al. Processing and cryopreservation of placental/umbilical cord blood for unrelated bone marrow reconstitution. Proc Natl Acad Sci USA 92:10119-10122). for subsequent cellular therapy. CD105 cells are expanded by culture and may be utilized for methods of the disclosure. CD105+ cells are purified from the mononuclear cell fraction by immuno-magnetic separation using the Magnetic Activated Cell Sorting (MACS) CD34+ Progenitor Cell Isolation Kit (Miltenyi-Biotec, Auburn, Calif.) according to manufacturers recommendations. The purity of the CD34+ cells obtained ranges between 95% and 98%, based on Flow Cytometry evaluation (FACScan flow cytometer, Becton-Dickinson, Immunofluorometry systems, Mountain View, Calif.). Cells are plated at a concentration of 10.sup.4 cells/ml in a final volume of 0.5 ml in 24 well culture plates (Falcon; Becton Dickinson Biosciences) in DMEM supplemented with the cytokine cocktail of: 20 ng/ml IL-3, 250 ng/ml IL-6, 10 ng/ml SCF, 250 ng/ml TPO and 100 ng/ml flt-3L and a 50% mixture of LPCM. LPCM is generated by obtaining a fresh human placenta from vaginal delivery and placing it in a sterile plastic container. The placenta is rinsed with an anticoagulant solution comprising phosphate buffered saline (Gibco-Invitrogen, Grand Island, N.Y.), containing a 1:1000 concentration of heparin (1% w/w) (American Pharmaceutical Partners, Schaumburg, Ill.). The placenta is then covered with a DMEM media (Gibco) in a sterile container such that the entirety of the placenta is submerged in said media, and incubated at 37° C. in a humidified 5% CO₂ incubator for 24 hours. At the end of the 24 hours, the live placenta conditioned medium (LPCM) is isolated from the container and sterile-filtered using a commercially available sterile 0.2 micron filter (VWR). Cells are expanded, checked for purity using CD34-specific flow cytometry and immunologically matched to recipients using a mixed lymphocyte reaction. Cells eliciting a low level of allostimulatory activity to recipient lymphocytes are selected for transplantation. Cells are administered as described above. Patients in the treated group display an increased responsiveness to insulin starting 2 weeks after injection of cells.

### Example 2

### Increased Insulin Responsiveness after Allogeneic Fibroblast Regenerative Cell

Embodiments of the disclosure include methods of increasing insulin responsiveness and/or insulin sensitivity upon administration of fibroblasts, including particular fibroblasts. In specific embodiments the fibroblasts are CD34+ and/or CD133+.

A group of 100 patients are having Type 2 diabetes and receiving daily insulin injections are utilized in a study. Fifty patients are treated with placebo control and 50 receive allogeneic skin-derived fibroblasts, as examples of fibroblasts. Patients in the treated group display an increased responsiveness to insulin, for example starting 1, 2, 3, 4, or more weeks after injection of cells.

### Example 3

### Intravenous Administration of Fibroblasts Increases Glucose Tolerance

C57BL/6 mice at 5 weeks of age were fed a high fat diet (HFD) in which 60% of calories came from fat, and for a total of 24 weeks. At 23 weeks of HFD feeding, mice were injected with 40 mg/kg streptozocin (Sigma-Aldrich, St. Louis, Mo., USA) daily for 3 consecutive days. Streptozocin kills pancreatic cells, resulting in a model of diabetes, including Type 2 Diabetes (T2D) in the mice.

Human dermal fibroblasts, bone marrow mesenchymal stem cells (BM-MSC) or adipose MSC, passage 3, were cultured in Opti-MEM media with 10% fetal calf serum. Cells were washed in phosphate buffered saline (PBS) and infused intravenously (5×10⁵/mouse, in 0.2 mL PBS) via the tail vein into the T2D recipients generated by HFD and STZ injections after 24 weeks of HFD. Groups of 10 mice per treatment were used.

Non-fasting blood glucose levels were measured daily using the Freestyle Lite blood glucometer (Abbott). Intraperitoneal glucose tolerance test was performed by administration of (2 g/kg glucose) 2 weeks after cell infusion with blood samples taken at baseline (0 min) and 30 and 120 minutes. A significantly improved control of blood glucose was demonstrated in animals treated with fibroblasts as compared to the two other types of MSC.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents and Patent Applications

U.S. Pat. No. 3,933,830

U.S. Pat. No. 3,950,518

U.S. Pat. No. 3,954,784

U.S. Pat. No. 3,961,065

U.S. Pat. No. 3,987,172

U.S. Pat. No. 3,992,388

U.S. Pat. No. 4,008,245

U.S. Pat. No. 4,042,583

U.S. Pat. No. 4,044,015

U.S. Pat. No. 4,052,507

U.S. Pat. No. 4,073,910

U.S. Pat. No. 4,093,616

U.S. Pat. No. 4,129,567

U.S. Pat. No. 4,141,898

U.S. Pat. No. 4,163,745

U.S. Pat. No. 4,172,835

U.S. Pat. No. 4,195,094

U.S. Pat. No. 4,207,330

U.S. Pat. No. 4,220,650

U.S. Pat. No. 4,262,018

U.S. Pat. No. 4,374,148

U.S. Pat. No. 4,305,955

U.S. Pat. No. 4,336,379

U.S. Pat. No. 4,336,391

U.S. Pat. No. 4,405,625

U.S. Pat. No. 4,419,353

U.S. Pat. No. 4,421,752

U.S. Pat. No. 4,430,337

U.S. Pat. No. 4,448,971

U.S. Pat. No. 4,467,681

U.S. Pat. No. 4,499,279

U.S. Pat. No. 4,622,406

U.S. Pat. No. 4,681,898

U.S. Pat. No. 4,695,634

U.S. Pat. No. 4,716,163

U.S. Pat. No. 4,740,521

U.S. Pat. No. 4,812,471

U.S. Pat. No. 4,816,484

U.S. Pat. No. 4,845,094

U.S. Pat. No. 4,845,231

U.S. Pat. No. 4,968,707

U.S. Pat. No. 5,019,580

U.S. Pat. No. 5,258,382

U.S. Pat. No. 5,268,373

U.S. Pat. No. 5,294,708

U.S. Pat. No. 5,420,146

U.S. Pat. No. 5,424,406

U.S. Pat. No. 5,444,086

U.S. Pat. No. 5,468,762

U.S. Pat. No. 5,480,896

U.S. Pat. No. 5,510,360

U.S. Pat. No. 5,531,991

U.S. Pat. No. 5,532,256

U.S. Pat. No. 5,545,672

U.S. Pat. No. 5,691,386

U.S. Pat. No. 5,674,900

U.S. Pat. No. 5,677,342

U.S. Pat. No. 5,681,958

U.S. Pat. No. 5,708,012

U.S. Pat. No. 5,728,720

U.S. Pat. No. 5,731,292

U.S. Pat. No. 5,747,527

U.S. Pat. No. 5,753,790

U.S. Pat. No. 5,756,513

U.S. Pat. No. 5,783,556

U.S. Pat. No. 5,817,634

U.S. Pat. No. 5,830,434

U.S. Pat. No. 5,830,873

U.S. Pat. No. 5,837,255

U.S. Pat. No. 5,849,989

U.S. Pat. No. 5,885,980

U.S. Pat. No. 5,900,240

U.S. Pat. No. 5,916,555

U.S. Pat. No. 5,925,647

U.S. Pat. No. 5,925,656

U.S. Pat. No. 5,929,055

U.S. Pat. No. 5,955,481

U.S. Pat. No. 5,980,902

U.S. Pat. No. 5,981,510

U.S. Pat. No. 5,990,139

U.S. Pat. No. 6,030,990

U.S. Pat. No. 6,037,359

U.S. Pat. No. 6,048,842

U.S. Pat. No. 6,057,343

U.S. Pat. No. 6,121,282

U.S. Pat. No. 6,903,073

U.S. Pat. No. 6,967,019

U.S. Pat. No. 7,033,831

U.S. Pat. No. 7,056,734

U.S. Pat. No. 7,138,275

U.S. Pat. No. 7,169,608

U.S. Patent Application No. 2005/0054093

### PUBLICATIONS

1. Paoletti, R., et al., Metabolic syndrome, inflammation and atherosclerosis. Vasc Health Risk Manag, 2006. 2(2): p. 145-52.
2. Scheen, A. J., Drug-drug and food-drug pharmacokinetic interactions with new insulinotropic agents repaglinide and nateglinide. Clin Pharmacokinet, 2007. 46(2): p. 93-108.
3. Joshi, S. R., Metformin: old wine in new bottle-evolving technology and therapy in diabetes. J Assoc Physicians India, 2005. 53: p. 963-72.
4. Goodarzi, M. O. and M. Bryer-Ash, Metformin revisited: re-evaluation of its properties and role in the pharmacopoeia of modern antidiabetic agents. Diabetes Obes Metab, 2005. 7(6): p. 654-65.
5. Eriksson, A., et al., Short-term effects of metformin in type 2 diabetes. Diabetes Obes Metab, 2007. 9(3): p. 330-6.
6. Green, B. D., et al., Inhibition of dipeptidyl peptidase-IV activity by metformin enhances the antidiabetic effects of glucagon-like peptide-1. Eur J Pharmacol, 2006. 547(1-3): p. 192-9.
7. Faveeuw, C., et al., Peroxisome proliferator-activated receptor gamma activators inhibit interleukin-12 production in murine dendritic cells. FEBS Lett, 2000. 486(3): p. 261-6.
8. Saubermann, L. J., et al., Peroxisome proliferator-activated receptor gamma agonist ligands stimulate a Th2 cytokine response and prevent acute colitis. Inflamm Bowel Dis, 2002. 8(5): p. 330-9.
9. Thomas, M. K., et al., Hedgehog signaling regulation of insulin production by pancreatic beta-cells. Diabetes, 2000. 49(12): p. 2039-47.
10. Oh, S. H., et al., Adult bone marrow-derived cells trans-differentiating into insulin-producing cells for the treatment of type I diabetes. Lab Invest, 2004. 84(5): p. 607-17.
11. Donath, M. Y., et al., Mechanisms of beta-cell death in type 2 diabetes. Diabetes, 2005. 54 Suppl 2: p. S108-13.
12. Brand, S. J., et al., Pharmacological treatment of chronic diabetes by stimulating pancreatic beta-cell regeneration with systemic co-administration of EGF and gastrin. Pharmacol Toxicol, 2002. 91(6): p. 414-20.
13. Suarez-Pinzon, W. L., et al., Combination therapy with epidermal growth factor and gastrin induces neogenesis of human islet {beta}-cells from pancreatic duct cells and an increase in functional {beta}-cell mass. J Clin Endocrinol Metab, 2005. 90(6): p. 3401-9.
14. Suarez-Pinzon, W. L., et al., Combination therapy with epidermal growth factor and gastrin increases beta-cell mass and reverses hyperglycemia in diabetic NOD mice. Diabetes, 2005. 54(9): p. 2596-601.
15. DeFronzo, R. A., Lilly lecture 1987. The triumvirate: beta-cell, muscle, liver. A collusion responsible for NIDDM. Diabetes, 1988. 37(6): p. 667-87.
16. Cheetham, C., et al., Losartan, an angiotensin type I receptor antagonist, improves conduit vessel endothelial function in Type II diabetes. Clin Sci (Lond), 2001. 100(1): p. 13-7.
17. Kajiguchi, M., et al., Safety and efficacy of autologous progenitor cell transplantation for therapeutic angiogenesis in patients with critical limb ischemia. Circ J, 2007. 71(2): p. 196-201.
18. Tsai, P. T., et al., A critical role of erythropoietin receptor in neurogenesis and post-stroke recovery. J Neurosci, 2006. 26(4): p. 1269-74.
19. Ogueta, S., et al., Prolactin is a component of the human synovial liquid and modulates the growth and chondrogenic differentiation of bone marrow-derived mesenchymal stem cells. Mol Cell Endocrinol, 2002. 190(1-2): p. 51-63.
20. Banerjee, M., M Kanitkar, and R. R. Bhonde, Approaches towards endogenous pancreatic regeneration. Rev Diabet Stud, 2005. 2(3): p. 165-76.
21. Wang, C., et al., Mechanical, cellular, and molecular factors interact to modulate circulating endothelial cell progenitors. Am J Physiol Heart Circ Physiol, 2004. 286(5): p. H1985-93.
22. Yildirim, S., et al., Expansion of cord blood CD34+ hematopoietic progenitor cells in coculture with autologous umbilical vein endothelial cells (HUVEC) is superior to cytokine-supplemented liquid culture. Bone Marrow Transplant, 2005. 36(1): p. 71-9.
23. Ablin, J. N., et al., Effect of anti-TNFalpha treatment on circulating endothelial progenitor cells (EPCs) in rheumatoid arthritis. Life Sci, 2006. 79(25): p. 2364-9.
24. Bossolasco, P., et al., Molecular and phenotypic characterization of human amniotic fluid cells and their differentiation potential. Cell Res, 2006. 16(4): p. 329-36.
25. Sartore, S., et al., Amniotic mesenchymal cells autotransplanted in a porcine model of cardiac ischemia do not differentiate to cardiogenic phenotypes. Eur J Cardiothorac Surg, 2005. 28(5): p. 677-84.
26. Prusa, A. R., et al., Neurogenic cells in human amniotic fluid. Am J Obstet Gynecol, 2004. 191(1): p. 309-14.
27. Tsai, M. S., et al., Clonal amniotic fluid-derived stem cells express characteristics of both mesenchymal and neural stem cells. Biol Reprod, 2006. 74(3): p. 545-51.
28. Forbes, S. J. and M. R. Alison, Side population (SP) cells: taking center stage in regeneration and liver cancer? Hepatology, 2006. 44(1): p. 23-6.
29. Meissner, K., et al., The ATP-binding cassette transporter ABCG2 (BCRP), a marker for side population stem cells, is expressed in human heart. J Histochem Cytochem, 2006. 54(2): p. 215-21.
30. Asakura, A. and M. A. Rudnicki, Side population cells from diverse adult tissues are capable of in vitro hematopoietic differentiation. Exp Hematol, 2002. 30(11): p. 1339-45.
31. Hierlihy, A. M., et al., The post-natal heart contains a myocardial stem cell population. FEBS Lett, 2002. 530(1-3): p. 239-43.
32. Rasmussen, B. B., et al., Insulin resistance of muscle protein metabolism in aging. Faseb J, 2006. 20(6): p. 768-9.
33. Scheen, A. J., Diabetes mellitus in the elderly: insulin resistance and/or impaired insulin secretion? Diabetes Metab, 2005. 31 Spec No 2: p. 5S27-5S34.
34. Borst, S. E. and G. J. Bagby, Neutralization of tumor necrosis factor reverses age-induced impairment of insulin responsiveness in skeletal muscle of Sprague-Dawley rats. Metabolism, 2002. 51(8): p. 1061-4.
35. Edelberg, J. M., et al., Young adult bone marrow-derived endothelial precursor cells restore aging-impaired cardiac angiogenic function. Circ Res, 2002. 90(10): p. E89-93.
36. Rubinstein, P., et al., Processing and cryopreservation of placental/umbilical cord blood for unrelated bone marrow reconstitution. Proc Natl Acad Sci USA, 1995. 92(22): p. 10119-22.
37. Pickup, J. C., Inflammation and activated innate immunity in the pathogenesis of type 2 diabetes. Diabetes Care, 2004. 27(3): p. 813-23.
38. dKern, P. A., et al., Adipose tissue tumor necrosis factor and interleukin-6 expression in human obesity and insulin resistance. Am J Physiol Endocrinol Metab, 2001. 280(5): p. E745-51.
39. Winkler, G., et al., Expression of tumor necrosis factor (TNF)-alpha protein in the subcutaneous and visceral adipose tissue in correlation with adipocyte cell volume, serum TNF-alpha, soluble serum TNF-receptor-2 concentrations and C-peptide level. Eur J Endocrinol, 2003. 149(2): p. 129-35.
40. Krogh-Madsen, R., et al., Influence of TNF-alpha and IL-6 infusions on insulin sensitivity and expression of IL-18 in humans. Am J Physiol Endocrinol Metab, 2006. 291(1): p. E108-14.
41. Derosa, G., et al., Telmisartan and irbesartan therapy in type 2 diabetic patients treated with rosiglitazone: effects on insulin-resistance, leptin and tumor necrosis factor-alpha. Hypertens Res, 2006. 29(11): p. 849-56.
42. Hotamisligil, G. S., et al., IRS-1-mediated inhibition of insulin receptor tyrosine kinase activity in TNF-alpha-and obesity-induced insulin resistance. Science, 1996. 271(5249): p. 665-8.
43. Ofei, F., et al., Effects of an engineered human anti-TNF-alpha antibody (CDP571) on insulin sensitivity and glycemic control in patients with NIDDM. Diabetes, 1996. 45(7): p. 881-5.
44. Dominguez, H., et al., Metabolic and vascular effects of tumor necrosis factor-alpha blockade with etanercept in obese patients with type 2 diabetes. J Vasc Res, 2005. 42(6): p. 517-25.
45. Moschen, A. R., et al., Visfatin, an adipocytokine with proinflammatory and immunomodulating properties. J Immunol, 2007. 178(3): p. 1748-58.
46. Janowska, J., B. Zahorska-Markiewicz, and M. Olszanecka-Glinianowicz, Relationship between serum resistin concentration and proinflammatory cytokines in obese women with impaired and normal glucose tolerance. Metabolism, 2006. 55(11): p. 1495-9.
47. Tam, L. S., et al., Impact of TNF inhibition on insulin resistance and lipids levels in patients with rheumatoid arthritis. Clin Rheumatol, 2007.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of treating or preventing insulin resistance in an individual, comprising the step of delivering to the individual a therapeutically effective amount of fibroblast cells.

2. The method of claim 1, wherein the fibroblasts are CD105+, CD34+, CD133+, or a mixture thereof.

3. The method of claim 1, wherein the fibroblasts are CD90+, CD45− and/or CD14−.

4. The method of claim 1, wherein the fibroblasts have regenerative activity.

5. The method of claim 4, wherein the fibroblasts have been exposed to erythropoietin, prolactin, human chorionic gonadotropin, gastrin, EGF, FGF, and/or VEGF.

6. The method of claim 1, wherein the insulin resistance is the result of diabetes, aging, low grade inflammation, obesity, pregnancy, metabolic syndrome X, congenital abnormality, or a combination thereof.

7. The method of claim 1, wherein the fibroblasts are derived from cord blood, peripheral blood, menstrual blood, placental matrix, endometrium, umbilical cord blood, deciduous teeth, muscle tissue, placenta, skin, bone marrow, amniotic fluid, adipose, umbilical cord matrix, omentum, subintestinal mucosa, or a mixture thereof.

8. The method of claim 1, wherein the fibroblast cells possess the ability to proliferate at a rate of more than one double per 24 hours when cultured at a concentration of 20,000 cells per well in a 96 well plate in 10% fetal calf serum in DMEM media.

9. The method of claim 1, wherein the fibroblast cells are delivered to the individual systemically or locally.

10. The method of claim 1, wherein the fibroblast cells are delivered to the individual intramuscularly.

11. The method of claim 1, wherein the fibroblast cells are delivered to the individual into or near the pancreas.

12. The method of claim 1, further comprising the step of providing to the individual a therapeutically effective amount of one or more anti-inflammatory agents.

13. The method of claim 1, further comprising the step of providing to the individual a therapeutically effective amount of one or more diabetes therapies.

14. A method of reducing blood glucose levels in an individual in need thereof, comprising the step of delivering to the individual a therapeutically effective amount of fibroblast cells.

15. The method of claim 14, wherein the fibroblasts are CD105+, CD34+, CD133+, or a mixture thereof.

16. The method of claim 14, wherein the fibroblasts are CD90+, CD45− and/or CD14−.

17. The method of claim 14, wherein the fibroblasts have regenerative activity.

18. The method of claim 17, wherein the fibroblasts have been exposed to erythropoietin, prolactin, human chorionic gonadotropin, gastrin, EGF, FGF, and/or VEGF.

19. The method of claim 14, wherein the individual has diabetes, is elderly, has low grade inflammation, is obese, is pregnant, has metabolic syndrome X, has a congenital abnormality, or a combination thereof.

20. The method of claim 14, wherein the fibroblasts are derived from cord blood, peripheral blood, menstrual blood, placental matrix, endometrium, umbilical cord blood, deciduous teeth, muscle tissue, placenta, skin, bone marrow, amniotic fluid, adipose, umbilical cord matrix, omentum, subintestinal mucosa, or a mixture thereof.

21. The method of claim 14, wherein the fibroblast cells possess the ability to proliferate at a rate of more than one double per 24 hours when cultured at a concentration of 20,000 cells per well in a 96 well plate in 10% fetal calf serum in DMEM media.

22. The method of claim 14, wherein the fibroblast cells are delivered to the individual systemically or locally.

23. The method of claim 14, wherein the fibroblast cells are delivered to the individual intramuscularly.

24. The method of claim 14, wherein the fibroblast cells are delivered to the individual into or near the pancreas.

25. The method of claim 14, further comprising the step of providing to the individual a therapeutically effective amount of one or more anti-inflammatory agents.

26. The method of claim 14, further comprising the step of providing to the individual a therapeutically effective amount of one or more diabetes therapies.
